(19) United States
(12) Patent Application Publication
(10) Pub. No.: US 2020/0093732 A1
(43) Pub. Date: Mar. 26, 2020

(54) FACIAL MASK COMPOSITION AND ITS PREPARATION METHOD

(71) Applicant: Shanghai Juku Cosmetic CO., LTD, Shanghai (CN)

(72) Inventor: Wen Liu, Shanghai (CN)

(21) Appl. No.: 16/138,755

(22) Filed: Sep. 21, 2018

(51) Int. Cl. A61K 8/9794; A61K 8/9789; A61K 8/9728; A61Q 19/00

(52) U.S. Cl. CPC A61K 8/9794; A61Q 19/007; A61K 8/9728; A61K 8/9789

(57) ABSTRACT

The present invention provides a facial mask composition, comprising by weight percentage, the following ingredients:
(a) 0.01% to 30% of emollient oil;
(b) 0.01% to 20% of moisturizer;
(c) 0.01% to 10% of thickening polymer;
(d) 0.01% to 10% of skin conditioner; and
(e) 10% to 90% of deionized water;
   wherein, the skin conditioner comprises, by weight parts: Radix Astragali 20, Aloe vera 30, Radix Angelicae Dahuricae 20 and Poria cocos 30.

### TECHNICAL FIELD

The present invention relates to a facial mask composition and its preparation method, falling into the manufacturing field of cosmetics.

### BACKGROUND

Some cosmetic masks in the prior art lack of skin conditioning ability in the case of ensuring moisturization, while other cosmetic masks have skin conditioning capability but lack of moisturization capability. Further, most of the existing skin conditioners are formed by chemicals, which inevitably harm the skin to some extent. In addition, chemical skin conditioners easily make the skin become dependent on it. Therefore, there is an urgent need for a product that can moisturize skin and condition skin by a pure natural Chinese medicine preparation, and its preparation method.

### SUMMARY OF THE INVENTION

The objective of the present invention is to provide a product that can moisturize skin and condition skin by a pure natural Chinese medicine preparation, and its preparation method.

In order to achieve above objective, the present invention adopts the following technical solution:

A facial mask composition, comprising, by weight percentage, the following ingredients:

(a) 0.01% to 30% of emollient oil;

(b) 0.01% to 20% of moisturizer;

(c) 0.01% to 10% of thickening polymer;

(d) 0.01% to 10% of skin conditioner; and

(e) 10% to 90% of deionized water;

wherein, the skin conditioner comprises, by weight parts: Radix Astragali 20, Aloe vera 30, Radix Angelicae Dahuricae 20 and Poria cocos 30.

The composition of the present invention may also comprise many kinds of other ingredients. Hereafter, the functions and effects of the specific ingredients of the present invention will be described in detail.

The emollient oil in the composition of the present invention can hold moisture, prevent excessive volatilization, and keep the skin moist and lustrous.

The content of emollient oil-cyclomethicone in the composition of the present invention ranges from about 0.01% to 20%, preferably from about 0.01% to 15%, and more preferably from about 0.01% to 10% based on the weight of the composition. The cyclomethicone is non-toxic, has no irritation to the skin and mucous membranes, and is well known to be safe. The cyclomethicone has excellent chemical stability, antistatic property and air permeability, so as to allow the skin to breathe freely, provide silky skin feel and reduce sticky feeling.

The content of emollient oil-caprylic/capric triglyceride in the composition of the invention ranges from about 0.01% to 20%, preferably from about 0.01% to 15%, and more preferably about 0.01% to 10% based on the weight of the composition. The caprylic/capric triglyceride has a low viscosity, is an excellent skin emollient, is easily to be absorbed, and endows the skin with silky luster.

The content of emollient oil-evening primrose oil in the composition of the present invention ranges from about 0.01% to 20%, preferably from about 0.01% to 15%, and more preferably from about 0.01% to 10% base on the weight of the composition. The evening primrose oil is extracted from natural plants, contains a variety of fatty acids, which mainly include linoleic acid, γ-linolenic acid, oleic acid, palmitic acid, stearic acid, Cis-6, 9,12-octadecatrienoic acid and cis-9,12,15-octadecatrienoic acid. The γ-linolenic acid contained therein is an essential fatty acid that the human body cannot synthesize per se, and has the effects of alleviating skin dryness, itching and even inflammation. Evening primrose oil also has the effects of resisting oxidation, moisturizing and softening skin, and is a good emollient.

Moisturizer

The moisturizer in the present invention with a content ranging from about 0.01% to 20% by weight is polyol, the composition of the present invention described herein preferably comprises the moisturizer with a content preferably ranging from about 1%-20%, and preferably from 2%-20%, the examples of the moisturizer include propylene glycol, sorbitol, butanediol, 1,2-hexanediol, caprylyl glycol, dipropylene glycol, glycerol, diglycerol, pentanediol, polyethylene glycol, and mixtures thereof. The glycerol and propylene glycol are preferably used in the composition of the present invention.

Another moisturizer-betaine in the present invention, with a scientific name of N-trimethylglycine, is a natural moisturizing factor. The betaine is a novel moisturizer with fast absorption and high activity. The betaine can rapidly improve the skin's moisture holding capacity, stimulate cell viability, and maintain skin moist and smooth.

Thickener

In some embodiments, the composition of the present invention may also include one or more thickeners, with a content ranging from about 0.01% to 10%, preferably from about 0.01% to 8%, and more preferably from about 0.01% to 6% based on the weight of the composition.

Non-limiting examples of the thickeners comprise those selected from the group consisting of carboxylic acid polymers (such as Carbopol 980), cross-linked polyacrylate polymers, synthetic high-molecular polymers (such as cellulose ethers), and natural high-molecular polymers (such as gum).

The acrylic polymer in the composition of the present invention, named as a copolymer of hydroxyethyl acrylate/sodium acryloyldimethyl taurate by CTFA, has excellent emulsifying and thickening ability, and endows the product with a soft texture.

The xanthan gum in the composition of the present invention is a monospore polysaccharide produced by fermentation of Pseudomonas spp., is a natural carbohydrate, and is an excellent thickener and stabilizer.

Hydroxyethyl cellulose in the composition of the present invention, which is a nonionic water-soluble cellulose ether, is a white or pale yellow, odorless and non-toxic powdery solid, and has the characteristics of a good thickening, suspension, dispersion, emulsification, adhesion, film formation, protecting moisture, and providing protective colloids.

Skin Conditioners

The various skin conditioners added into the composition of the present invention include, by weight parts, Radix Astragali 20, Aloe vera 30, Radix Angelicae Dahuricae 20 and Poria cocos 30.

Above Chinese medicine skin conditioner has the effects of whitening, shading, sterilizing and resisting corrosion, relieving pain, and resisting oxidation, can make the skin hold moisture and become moist and soft, and is a special-effect additive of cosmetics for cosmetology and hairdressing. The cosmetics with addition of Chinese medicine skin conditioner have the effects of protecting tissues, hydrophilicity, hygroscopicity and preventing moisture emission, etc.; and allow the skin to be soft and elastic and have a beautiful luster. The Chinese medicine composition can also promote tissue growth and cell metabolism, and soften the stratum corneum protein.

Normal human skin contains 10% to 20% of water in the stratum corneum, which has a great effect on moisturizing the skin, preventing skin aging and preventing various stimuli from external environment. With the increasing of age or the change of the environment, the water content will decrease to below 10%, and the skin will become dry and wrinkles appear. The sodium hyaluronate in the composition of the present invention can solve this problem; the sodium hyaluronate has a theoretical water retention value reaching 1000 ml/g, and is a key water retention component in human skin; and the sodium hyaluronate not only has a good moisturizing effect, but also avoids the disadvantage of sticky feeling and pore clogging. The sodium hyaluronate can also eliminates oxygen radicals and repair damaged skin caused by sun exposure.

Further, the present invention provides a method for preparing above facial mask composition, comprising the following steps:

1) uniformly mixing the oil-phase ingredients to obtain an oil phase;

2) mixing the water and the aqueous ingredients, heating to 70° C.-90° C. until the aqueous ingredients are dissolved to obtain a water phase; and

3) adding the oil phase into the water phase when the temperature is 55° C.-65° C., homogenizing, and uniformly stirring; and adding other ingredients when the temperature is 50° C., sufficiently and uniformly stirring, and discharging the resultant.

Since the invention adopts above technical solution, after use, the mask composition opens permeation channels for the skin, and can whiten skin, deeply supplement water and infiltrate the nutrient substance. It can sufficiently moisturize skin at night, open deep penetration channels for skin, supplement amino acids, strengthen skin renewability, and allow skin to smoothly breathe.

### DETAILED DESCRIPTION OF THE EMBODIMENTS

The preparing process of the present invention is to mix all of the ingredients into a uniform mixture, and comprises the following steps:

1) uniformly mixing the oil-phase ingredients to obtain an oil phase;

2) mixing water, glycerol, hydroxyethyl acrylate/sodium acryloyldimethyl taurate copolymer, methylparaben, xanthan gum, hydroxyethyl cellulose and disodium EDTA, heating to 80° C. and uniformly dispersing them; and

3) adding the oil phase into the water phase when the temperature is 60° C., homogenizing, and uniformly stirring;

4) adding the Chinese medicine composition (comprising, by weight parts, Radix Astragali 20, Aloe vera 30, Radix Angelicae Dahuricae 20 and Poria cocos 30) when the temperature is 50° C., uniformly stirring, and discharging the resultant; and

5) storing→performing semifinished product inspection→filling→performing finished product inspection→placing into a warehouse.

### EXAMPLES

The products of Examples 1-4 were prepared according to Table 1.

Each of the products of Examples 1-4 is prepared by the following steps: mixing the ingredients of phase 1 in a container; placing the ingredients of phase 2 in another container and heating to 80° C.; adding the phase 1 into the phase 2 when the temperature is 60° C., performing emulsification, homogenizing, uniformly stirring; and cooling to 50° C., adding phase 3, uniformly stirring, and discharging the resultant.

Test Method:

In order to test the moisturizing and conditioning effects of the present invention, a trying survey experiment was conducted:

1. Survey respondents: 50 ordinary consumers in various regions are surveyed, and 46 questionnaires for the trying condition were obtained;

2. Survey time: Dec. 10, 2017-Feb. 10, 2018;

3. Used products: Product No. 1 is a product prepared according to the formulation of the present invention; and Product No. 2 is a similar product of a competing manufacturer;

4. Test method: After a tester's face was cleaned, the Product No. 1 and the Product No. 2 were applied onto the left and right cheeks of the tester respectively to observe and feel the using effects;

5. A test table is showed as follows:

Table 2 Feedback summary after using Product No. 1 (60 days)

Table 3 Feedback summary after using Product No. 2 (60 days)

Table 4 Comparison statistical result of the conditioning effects of Product No. 1 and Product No. 2

6. Feedback conclusion:

All of the testers fedback that, the Product No. 1 can moisturize and is not overly thick, and can allow the skin to be moist and to freely breathe. After frequent use, the skin color significantly became whiter, ruddy and lustrous; wrinkles were significantly reduced, the skin became white and tender; and none of the questionnaires indicated the tester suffered from anaphylaxis.

| | | | | |
|---|---|---|---|---|
| | Example 1 | Example 2 | Example 3 | Example 4 |
| Ingredients | (wt. %) | (wt. %) | (wt. %) | (wt. %) |

Phase 1

| | | | | |
|---|---|---|---|---|
| Cyclomethicone | 4.0 | 4.0 | 5.0 | 5.0 |
| Caprylic/capric | 2.0 | 2.0 | 3.0 | 3.0 |
| triglyceride | | | | |
| Evening primrose oil | 3.0 | 3.0 | 2.0 | 2.0 |
| Essence | 0.2 | 0.2 | 0.2 | 0.2 |

Phase 2

| | | | | |
|---|---|---|---|---|
| deionized water | 46.7 | 51.2 | 48.3 | 48.3 |
| Glycerol | 20.0 | 20.0 | 15.0 | 15.0 |
| Butanediol | 4.0 | — | — | 4.0 |
| Hydroxyethyl acrylate/ | 3.0 | 3.0 | 3.0 | — |
| sodium acryloyldimethyl | | | | |
| taurate copolymer | | | | |
| Methylparaben | 0.2 | 0.2 | 0.2 | 0.2 |
| Xanthan gum | 0.3 | 0.3 | 0.3 | 0.3 |
| Hydroxyethyl cellulose | 0.2 | 0.2 | 0.1 | 0.1 |
| Disodium EDTA | 0.1 | 0.1 | 0.1 | 0.1 |

Phase 3

| | | | | |
|---|---|---|---|---|
| Sodium hyaluronate | 0.5 | 0.5 | 1.0 | 1.0 |
| deionized water | 5.0 | 5.0 | 10.0 | 10.0 |
| Dipotassium | 1.0 | 0.5 | 0.5 | 1.0 |
| glycyrrhizinate | | | | |
| Radix Astragali | — | 2.0 | 2.0 | 2.0 |
| Aloe vera | 2.0 | 2.0 | 2.0 | — |
| Radix Angelicae | 2.0 | — | 2.0 | 2.0 |
| Dahuricae | | | | |
| Betaine | 4.0 | 4.0 | 4.0 | 4.0 |
| Poria cocos | 0.5 | 0.5 | — | 0.5 |
| Glycolyurea/ | 0.3 | 0.3 | 0.3 | 0.3 |
| Iodopropynyl | | | | |
| butylcarbamate | | | | |

| | Evaluation effect | | | | | | |
|---|---|---|---|---|---|---|---|
| | (by the number of testers) | | | | | | |
| | Very | | | | Very | | |
| Evaluation item | good | Good | General | Poor | poor | Unknown | Else |
| Product | 39 | 6 | 1 | 0 | 0 | 0 | |
| appearance | | | | | | | |
| Product odor | 33 | 6 | 7 | 0 | 0 | 0 | |
| type | | | | | | | |
| Product | 35 | 8 | 3 | 0 | 0 | 0 | |
| thickness | | | | | | | |
| Product | 35 | 8 | 3 | 0 | 0 | 0 | |
| moisturizing | | | | | | | |
| effect | | | | | | | |
| Product sticky | 40 | 6 | 0 | 0 | 0 | 0 | |
| feeling | | | | | | | |
| Product | 30 | 10 | 6 | 0 | 0 | 0 | |
| nourishing | | | | | | | |
| effect | | | | | | | |

| | | | | |
|---|---|---|---|---|
| Anaphylaxis | Yes | 0 | No | 43 |

| | Evaluation effect | | | | | | |
|---|---|---|---|---|---|---|---|
| | (by the number of testers) | | | | | | |
| | Very | | | | Very | | |
| Evaluation item | good | Good | General | Poor | poor | Unknown | Else |
| Product | 40 | 3 | 0 | 0 | 0 | 0 | |
| appearance | | | | | | | |
| Product odor | 26 | 7 | 5 | 5 | 0 | 0 | |
| type | | | | | | | |
| Product | 35 | 8 | 0 | 0 | 0 | 0 | |
| thickness | | | | | | | |
| Product | 27 | 8 | 5 | 3 | 0 | 0 | |
| moisturizing | | | | | | | |
| effect | | | | | | | |
| Product sticky | 19 | 9 | 11 | 4 | 0 | 0 | |
| feeling | | | | | | | |
| Product | 22 | 10 | 10 | 3 | 0 | 0 | |
| nourishing | | | | | | | |
| effect | | | | | | | |

| | | | | |
|---|---|---|---|---|
| Anaphylaxis | Yes | 2 | No | 41 |

| | Evaluation of product conditioning effect | | | | | |
|---|---|---|---|---|---|---|
| | (by the number of testers) | | | | | |
| | Very good | Good | General | Poor | Very poor | Unknown |
| Product 1 | 38 | 5 | 3 | 0 | 0 | 0 |
| Product 2 | 25 | 12 | 8 | 1 | 0 | 0 |

1. A facial mask composition comprising, by weight percentage, the following ingredients:
(a) 0.01% to 30% of emollient oil;
(b) 0.01% to 20% of moisturizer;
(c) 0.01% to 10% of thickening polymer;
(d) 0.01% to 10% of skin conditioner; and
(e) 10% to 90% of deionized water;
wherein, the skin conditioner comprises, by weight parts: Radix Astragali 20, Aloe vera 30, Radix Angelicae Dahuricae 20 and Poria cocos 30.

2. The facial mask composition according to claim 1, comprising by weight percentage, the following ingredients:
(a) 1% to 20% of emollient oil;
(b) 1% to 20% of moisturizer;
(c) 0.01% to 8% of thickening polymer; and
(d) 0.01% to 8% of skin conditioner;
wherein, the skin conditioner comprises, by weight parts: Radix Astragali 20, Aloe vera 30, Radix Angelicae Dahuricae 20 and Poria cocos 30.

3. The facial mask composition according to claim 1, comprising the following ingredients, by weight percentage:
(a) 5% to 15% of emollient oil;
(b) 5% to 20% of moisturizer;
(c) 0.01% to 6% of thickening polymer; and
(d) 0.01% to 6% of skin conditioner;
wherein, the skin conditioner comprises, by weight parts: Radix Astragali 20, Aloe vera 30, Radix Angelicae Dahuricae 20 and Poria cocos 30.

4. The facial mask composition according to claim 1, wherein, the emollient oil is one or more selected from cyclomethicone, caprylic/capric triglyceride, and evening primrose oil.

5. The facial mask composition according to claim 1, wherein, the moisturizer is one or more selected from betaine, propylene glycol, sorbitol, butanediol, 1,2-hexanediol, 1,2-caprylyl glycol, dipropylene glycol, glycerol, diglycerol, pentylene glycol and polyethylene glycol; preferably is betaine, glycerol or propylene glycol.

6. The facial mask composition according to claim 1, wherein, the thickening polymer is one or more selected from polysaccharides, acrylic copolymers and cellulosic polymers; preferably is one or more of hydroxyethyl acrylate/sodium acryloyldimethyl taurate copolymer named by CTFA, xanthan gum, and hydroxyethyl cellulose.

7. A method for preparing the facial mask according to claim 1, comprising the following steps:
1) uniformly mixing the oil-phase ingredients to obtain an oil phase;
2) mixing the water and the aqueous ingredients, heating to 70° C.-90° C. until the aqueous ingredients are dissolved to obtain a water phase; and
3) adding the oil phase into the water phase when the temperature is 55° C.-65° C., homogenizing, and uniformly stirring; and adding the skin conditioner when the temperature is 50° C., sufficiently and uniformly stirring, and discharging the resultant.
